(19) United States
(12) Patent Application Publication
(10) Pub. No.: US 2023/0343412 A1
(43) Pub. Date: Oct. 26, 2023

(54) METHOD, DEVICE, AND MEDIUM FOR RESULT PREDICTION FOR ANTIBODY SEQUENCE

(71) Applicants: Beijing Youzhuju Network Technology Co., Ltd, Beijing (CN); Lemon Inc., Grand Cayman (KY)

(72) Inventors: Fei YE, Beijing (CN); Danqing WANG, Beijing (CN)

(21) Appl. No.: 18/058,611

(22) Filed: Nov. 23, 2022

(51) Int. Cl. G16B 20/30; G16B 20/50

(52) U.S. Cl. CPC G16B 20/30; G16B 20/50

(57) ABSTRACT

Systems and methods directed to providing a method for determining a prediction result related to an antibody sequence. The method comprises obtaining an antibody sequence comprising a plurality of amino acids, and obtaining a germline sequence of the antibody sequence. The method further comprises determining a prediction result related to the antibody sequence based on at least one of: evolution information between the antibody sequence and the germline sequence, or a mutation position on the antibody sequence, wherein an amino acid of the plurality of amino acids mutates on the mutation position.

### BACKGROUND

Antibodies are vital proteins produced by the immune system offering robust protection for human body from harmful pathogens. Antibodies have been extensively used for researching diverse diseases, such as SARS-CoV-2. To perform protective function, antibody sequence undergoes evolution selection to search for optimal patterns that can specifically recognize pathogens. Deciphering the information stored in antibody sequences can benefit understanding of disease and accelerate therapeutic antibody development.

Recent advent of high-throughput sequencing has led to an exponential increase in unlabeled antibody sequences. For example, the total number of antibody sequences has doubled from 70 million to 1.5 billion in the last five years, providing a gold mine for data-driven unsupervised learning. In addition, large-scale pre-training language models have shown powerful strength in extracting information from massive unlabeled sequences, offering a unique opportunity to learn the general biological semantics for antibody sequences. An example of learning the general biological semantics for antibody sequences is representation learning for antibody analysis. And antibody representation learning has attracted increasing interest for its potential strength to benefit the fundamental understanding of disease and therapeutic antibody development.

### SUMMARY

In accordance with examples of the present disclosure, a method for determining a prediction result related to an antibody sequence is described. The method comprises obtaining an antibody sequence including a plurality of amino acids. The method further comprises obtaining a germline sequence of the antibody sequence. The method further comprises determining a prediction result related to the antibody sequence based on at least one of: evolution information between the antibody sequence and the germline sequence, or a mutation position on the antibody sequence, wherein an amino acid of the plurality of amino acids mutates on the mutation position.

In accordance with examples of the present disclosure, an electronic device comprising a memory and a processor is described. In examples, the memory is used to store one or more computer instructions which, when executed by the processor, cause the processor to: obtain an antibody sequence including a plurality of amino acids; obtain a germline sequence of the antibody sequence; and determine a prediction result related to the antibody sequence based on at least one of: evolution information between the antibody sequence and the germline sequence, or a mutation position on the antibody sequence, wherein an amino acid of the plurality of amino acids mutates on the mutation position.

A non-transitory computer-readable medium including instructions stored thereon which, when executed by an apparatus, cause the apparatus to perform acts including: obtaining an antibody sequence including a plurality of amino acids; obtaining a germline sequence of the antibody sequence; and determining a prediction result related to the antibody sequence based on at least one of: evolution information between the antibody sequence and the germline sequence, or a mutation position on the antibody sequence, wherein an amino acid of the plurality of amino acids mutates on the mutation position.

Any of the one or more above aspects may be in combination with any other of the one or more aspects. Any of the one or more aspects as described herein.

This Summary is provided to introduce a selection of concepts in a simplified form, which is further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the following description and, in part, will be apparent from the description, or may be learned by practice of the disclosure.

### BRIEF DESCRIPTION OF THE DRAWINGS

To easily identify the discussion of any particular element or act, the most significant digit or digits in a reference number refer to the figure number in which that element is first introduced.

FIG. 1 illustrates an exemplified process of evolution of antibody sequences in a B-cell.

FIG. 2 illustrates a flowchart of a method for determining a prediction result related to an antibody sequence.

FIG. 3 illustrates an exemplified EATLM model for implementing B-cell classification.

FIG. 4 illustrates prediction results of multiple antibody sequences on each B-cell category.

FIG. 5 illustrates an exemplified EATLM model for implementing a disease classification prediction.

FIG. 6 illustrates an exemplified EATLM model for implementing antigen binding prediction.

FIG. 7 illustrates a flowchart of a method for training the EATLM model according to the present disclosure.

FIG. 8 illustrates a language model for a pre-training process according to embodiments of the present disclosure.

FIG. 9 is a block diagram illustrating physical components (e.g., hardware) of an electronic device with which aspects of the disclosure may be practiced.

### DETAILED DESCRIPTION

In the following detailed description, references are made to the accompanying drawings that form a part hereof, and in which are shown by way of illustrations specific aspects or examples. These aspects may be combined, other aspects may be utilized, and structural changes may be made without departing from the present disclosure. Aspects may be practiced as methods, systems or devices. Accordingly, aspects may take the form of a hardware implementation, an entirely software implementation, or an implementation combining software and hardware aspects. The following detailed description is therefore not to be taken in a limiting sense, and the scope of the present disclosure is defined by the appended claims and their equivalents.

Antibody representation learning has attracted increasing interest for its potential strength to benefit the fundamental understanding of disease and therapeutic antibody development. With the increase of the number of antibody sequences and the development of large-scale pre-training language models, antibody representation learning with antibody self-supervised pre-training antibody language models has achieved promising results in understanding the biological characteristics.

Researchers have developed some models to explore the capabilities of large-scale pre-training language models in extracting antibody information. For example, there is an increasing interest to explore large-scale language models using protein sequences. These models may be referred to be “protein language models” and may also have been shown to achieve state-of-art capacity in predicting protein mutations, structure, and protein evolution. Adopting transformer language models for protein sequence analysis has demonstrated that self-supervision is a promising paradigm for protein. However, the evolution of protein is fundamentally different from that of antibodies and the general representation of natural proteins cannot well fit into antibody-specific features. In addition, no work has systematically explored the role of protein language models for the task of antibody prediction.

For another example, researchers have been inspired by the protein language models, and are making an effort to develop antibody-specific language models for antibody function understanding. Antibody-specific language model has been developed. The model trained under multiple instance learning frameworks may identify antibody key binding residues for antigens. This work shows that the representation obtained from the language model is useful for antibody sequences clustering into trajectories resembling affinity maturation. Recently, an antibody language model, which may learn biology-meaning representations for antibodies, has been developed. The model may restore missing residues of antibody sequences better than the protein language models. However, these pre-training models simply treat the amino acid sequences of the antibody as a translation language and do not take into account any characteristics of the antibody when modeling, therefore, information about the antibody has not been effectively explored or learned, which may result in a relatively low accuracy of the prediction.

Therefore, it can be seen from above, although representation learning for antibody analysis has been rapidly developed, two key challenges impede the usage of representation learning for antibody analysis. First, despite several studies of antibody representation learning, a reliable benchmark for comprehensive performance evaluation is very much lacking, which certainly hinders future research in antibodies. Second, the existing methods view antibody sequences as texts and simply adopt masked language modeling for representation learning. Accordingly, this formulation of pre-training cannot sufficiently and efficiently extract antibody biological information, which is crucial for many antibody functions. It should be understood that, the information used in this disclosure is obtained with the consent of the user and is therefore legally obtained and the information used in this disclosure is obtained from an open-sourced material.

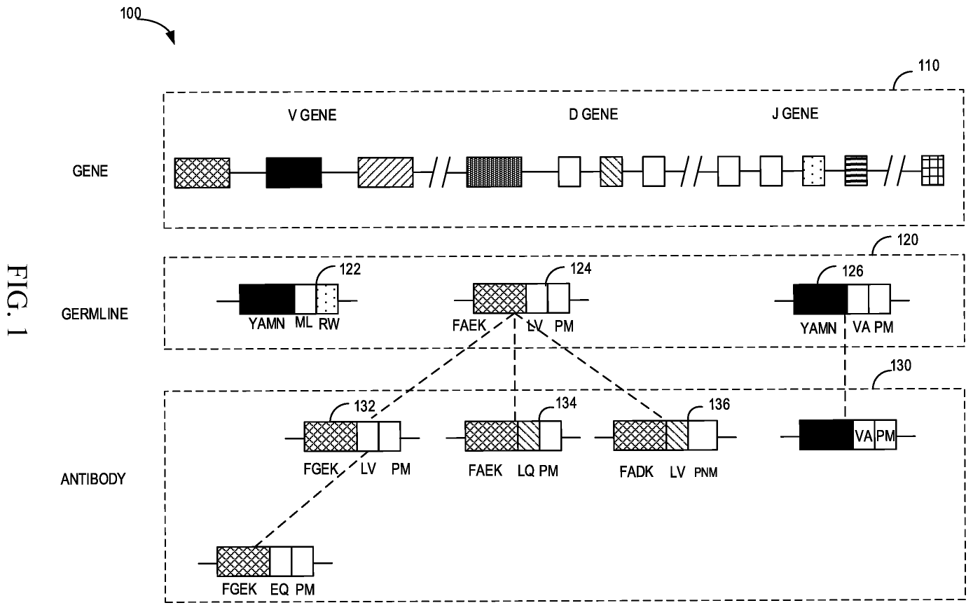

FIG. 1 illustrates an exemplified process 100 of evolution of antibody sequences in a B-cell. B-cell antibodies serve as critical barriers to viral infection. FIG. 1 illustrates the exemplified process 100 of evolution of antibody sequences in a B-cell. As shown in FIG. 1, in block 110, multiple genes segments, for example, V gene, D gene and J gene segments, are shown. The evolution starts from the random recombination of V gene, D gene and J gene segments to form multiple ancestor sequences which are germlines, as shown in block 120. For example, as shown in FIG. 1, three generated germlines 122, 124, and 126 are shown, for example. During antibody evolution from germlines, members of antibodies are descended from a common ancestor (i.e., a germline), which are diverse amino acid sequences generated by gene sequence recombination. In FIG. 1, the evolution sequence relationships are highlighted using dash lines. For example, as shown in FIG. 1, antibody sequences 132, 134, and 136 are descended from germline 124. Specifically, upon exposure to pathogens, a germline undergoes frequent mutations, termed somatic hypermutation. For example, as shown in FIG. 1, the amino acid “Alanine” (A) in germline 126 is mutated to be “Glycine” (G) in a mutation position in the antibody sequence 132. Mutation occurs in germlines to facilitate searching for progeny sequences with optimal binding specificity. In other words, the ancestor germline sequences undergo frequent sequence mutations to search for progeny sequences with an optimal binding capacity to specific pathogens.

As the complexity of the sequence relationship directly correlates with the antibody's binding specificity to certain antigens, sequence evolution analysis has been employed by many computational biology studies and shows promising results in antibody-related tasks, such as disease classification and therapeutic antibody development. Therefore, a combination of a large-scale language model and representation learning for antibody analysis by exploring antibody evolution information is desirable.

In some embodiments, a method of determining a prediction result related to an antibody sequence includes obtaining an antibody sequence comprising a plurality of amino acids. The method further includes obtaining a germline sequence of the antibody sequence. The method further includes determining a prediction result related to the antibody sequence based on at least one of: evolution information between the antibody sequence and the germline sequence, or a mutation position on the antibody sequence, wherein an amino acid of the plurality of amino acids mutates on the mutation position.

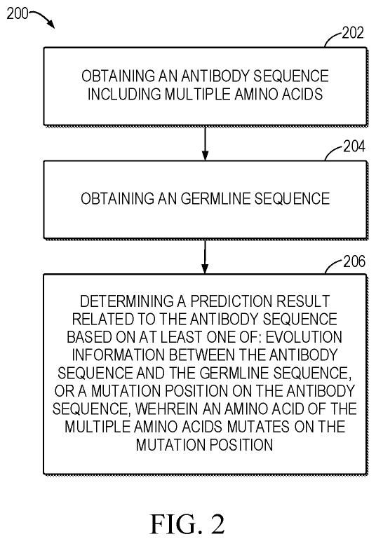

FIG. 2 illustrates a flowchart of a method 200 for determining a prediction result related to an antibody sequence. The method 200 of FIG. 2A may be carried out by any electronic device(s). It should be appreciated that some of the steps can be combined, omitted, performed in parallel, or performed in a different sequence, without affecting the functions achieved.

At 202, the electronic device obtains an antibody sequence including multiple amino acids. An example of an antibody sequence can be found from FIG. 1, e.g. an antibody sequence 132. The antibody sequence 132 includes multiple amino acids, such as Phenylalanine (F), Glycine (G), Glutamate (E), Lysine (K), Leucine (L), Valine (V), Proline (P), and Methionine (M). Similarly, antibody sequence 134 and antibody sequence 136 have similar structures including multiple amino acids and the detailed explanation of these amino acids is omitted herein for the purposes of clarity and brevity.

At 204, the electronic device obtains a germline sequence of the antibody sequence. In some embodiments, members of antibodies are descended from a common ancestor (i.e., a germline), which are diverse amino acid sequences generated by gene sequence recombination. For example, as shown in FIG. 1, the ancestor of the antibody sequences 132, 134, and 136 are descended from the common ancestor, that is, the germline sequence 124. The germline sequence 124 is generated by a random combination of V gene, D gene, and J gene segments, and also includes multiple amino acids.

For example, as shown in FIG. 1, the germline sequence 124 includes amino acids such as Phenylalanine (F), Alanine (A), Glutamate (E), Lysine (K), Leucine (L), Valine (V), Proline (P), and Methionine (M). It should be noted that, the germline sequence 124 and the antibody sequences 132, 134, and 136 are shown for illustrated purposes, and the germline sequence and the antibody sequences may include any other amino acids or any combination thereof.

In some embodiments, the input antibody sequence may be represented by A={a₁, a₂, . . . aₘ} and the germline sequence may be represented by G={g₁, g₂, . . . , gₙ}, where m and n are the lengths of the respective sequences. Each token aᵢ or gⱼ (1≤i≤m; 1≤j≤n) in the sequence is called a residue that belongs to the amino acid set A. A may include 20 common amino acids and additional ‘X’ that indicates the residue is unknown (mostly in the germline).

In some embodiments, the germline sequence may be obtained from an open-sourced database, for example, via basic local alignment search tool (igBLAST). The present disclosure does not limit the number of the obtained antibody sequences and the germline sequences, and the number of the obtained antibody sequences and the germline sequences may be varied according to applications and requirements of the electronic device implementing the method 200.

At 206, the electronic device determines a prediction result related to the antibody sequence based on at least one of: evolution information between the antibody sequence and the germline sequence, or a mutation position on the antibody sequence. In some embodiments, an amino acid of the multiple amino acids mutates on the mutation position. In other words, the mutation position is a position which an amino acid of the multiple amino acids mutates. Depending on the mutation, one or more mutation positions may occur on the antibody sequence. Accordingly, one or more amino acids mutates on the antibody sequence. The number of mutated amino acids is not limited in the present disclosure.

The evolution information between the antibody sequence and the germline sequence focuses on the evolutionary similarity between the antibody sequence and its ancestor germline sequence, and may be used to discriminate the evolutionary relationship between antibody and germline sequences. The mutation position on the antibody sequence highlights the differences between the antibody sequence and its ancestor germline sequence, and may predict mutation positions and residue by mimicking somatic hypermutation during the evolution. Since the relationship between the antibody sequence and its ancestor germline sequence implies the evolutionary process, which significantly affects the biological functions of antibodies, the method according to embodiments of the present disclosure utilizes the evolution information between the antibody sequence and its ancestor germline sequence. Therefore, the prediction result can be significantly improved.

The method 200 according to embodiments of the present disclosure may be used to quantitatively evaluate the performance of the antibody representation. In some embodiments, for example, six biologically relevant supervised tasks are included, requiring different levels of sequence understanding. These tasks comprehensively highlight three major aspects of antibody applications: B-cell analysis, disease classification, and therapeutic antibody development. Improvement on these tasks can further facilitate the scientific discovery of clinically useful antibodies against viruses. Remarkably, by implementing the method 200 according to embodiments of the present disclosure, 37 potential SARS-CoV-2 binders whose sequences are highly identical to therapeutic antibodies are known to bind the virus.

In some embodiments, the method 200 as shown in FIG. 2 may be implemented by a language model which is antibody evolution-aware. For a convenience of reference, the model implementing the method 200 will be referred to as evolution-aware antibody language model, “EATLM” model for short. In some embodiments, the EATLM model according to embodiments of the disclosure may include a transformer encoder. The transformer encoder may be implemented according to the existing technology or any technology to be developed in the future. The EATLM model may be used to perform the method 200 to determine a prediction result related to the antibody sequence based on at least one of: evolution information between the antibody sequence and the germline sequence, or a mutation position on the antibody sequence. In the following description, the detailed explanation of determining a prediction result related to the antibody sequence will be described in combination with the EATLM model.

In some embodiments, the prediction result related to the antibody sequence may include B-cell analysis; accordingly, the method 200 as shown in FIG. 2 may be used to implement B-cell analysis. B-Cell analysis includes the classification performance and analysis of B-cell classification. It is a 6-category classification task to distinguish the maturation stage of B-cell antibody sequences. Accordingly, the prediction result related to the antibody sequence may include a category that a B-cell belongs to. In some embodiments, the category is selected from at least one of: immature B-cell, transitional B-cell, mature B-cell, plasmacytes PC, memory IgD−, and memory IgD+. Different B-cell types correspond to different evolution stages in the immune system. That is, in the 6-category classification task of the B-cell analysis, each sequence may belong to one of {immature, transitional, mature, plasmacytes, memory, IgD+, memory IgD−}. By analyzing B-cell, it helps a better understanding of the mechanism of immune evolution, which is a critical biological process in the immune system affecting the function and antigen specificity of antibodies.

In some embodiments, the EATLM model according to embodiments of the present disclosure may be used to perform the method 200 to determine B-cell classification related to the antibody sequence based on at least one of: evolution information between the antibody sequence and the germline sequence, or a mutation position on the antibody sequence. Specifically, in some embodiments, the EATLM model according to embodiments of the present disclosure may determine a B-cell classification related to the antibody sequence based on evolution information between the antibody sequence and the germline sequence. In some embodiments, the EATLM model may determine a B-cell classification based on a mutation position on the antibody sequence. In some embodiments, the EATLM model may determine a B-cell classification based on evolution information between the antibody sequence and the germline sequence as well as on a mutation position on the antibody sequence. The mutation position is a position which an amino acid of the multiple amino acids mutates.

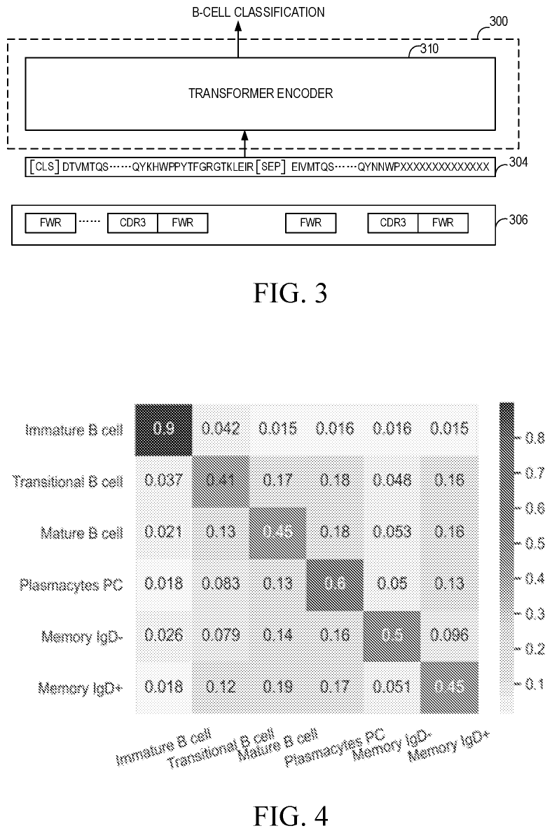

FIG. 3 illustrates an exemplified EATLM model for implementing B-cell classification. As shown in FIG. 3, the EATLM model 300 includes a transformer encoder for encoding an input of the EATLM model 300. As stated above, the transformer encoder may be implemented according to the existing technology or any technology to be developed in the future. The EATLM model 300 as shown in FIG. 3 may be used to perform the method 200 to determine a B-cell classification related to the antibody sequence based on at least one of: evolution information between the antibody sequence and the germline sequence, or a mutation position on the antibody sequence. In some embodiments, the EATLM model 300 as shown in FIG. 3 may determine a B-cell classification related to the antibody sequence based on evolution information between the antibody sequence and the germline sequence. In some embodiments, the EATLM model 300 may determine a B-cell classification based on a mutation position on the antibody sequence. In some embodiments, the EATLM model 300 may determine a B-cell classification based on evolution information between the antibody sequence and the germline sequence as well as on a mutation position on the antibody sequence. The mutation position is a position which an amino acid of the multiple amino acids mutates.

As shown in FIG. 3, the input of the EATLM model 300 may include an antibody sequence and a germline of the antibody sequence. It should be understood that, the number of antibody sequences are not limited in the embodiments of the present disclosure, and may be depended on the actual application and requirements of the EATLM model 300. An example of input antibody sequences and germlines of the antibody sequences is shown in FIG. 3, as labeled by 304. In some embodiments, the input antibody sequence and germline of the antibody sequence may be descended from a complementarity-determining region (e.g., CDR3) and/or a framework region (FWR) as shown in in FIG. 3, as labeled by 306. The EATLM model 300 may determine a B-cell classification related to the antibody sequence based on at least one of: evolution information between the antibody sequence and the germline sequence, or a mutation position on the antibody sequence.

FIG. 4 illustrates prediction results of multiple antibody sequences on each B-cell category. The order of B-cell type follows the evolutionary process in the immune system, which means that the immature B-cell first evolves to transitional B and then becomes a memory B-cell. Both memory IgD− and IgD+ belong to memory B-cells with different isotypes, and they have a high affinity to foreign antigens. Among the other categories, the Plasmacytes PC sequences also have some affinity ability. For each row in FIG. 4, the true category is on the left and the number indicates the probability for predicting another category. As shown in FIG. 4, the immature B-cell is easy to be classified with an accuracy of 0.9, and barely sequence will be misclassified into immature B-cell. FIG. 4 may indicate the EATLM model 300 according to embodiments of the present disclosure captures the specific characteristic of ancestor antibody sequences.

In addition, each pᵢⱼ in i-th row and j-th column in FIG. 4 indicates the probability for the EATLM model 300 according to embodiments of the present disclosure to predict a B-cell sequence in i category to j category. The number is normalized by row. According to the diagonal of the table as shown in FIG. 4, which has been shown with different grey scales, it may also be inferred that the EATLM model 300 according to embodiments of the present disclosure tends to mistake the B-cell sequences with their previous or post evolutionary stage, which is consistent with a biological process.

In some embodiments, the prediction result related to the antibody sequence may include a disease classification prediction; accordingly, the EATLM model may be used to perform the method 200 to determine a disease classification of the antibody sequence based on at least one of: evolution information between the antibody sequence and the germline sequence, or a mutation position on the antibody sequence. In the following description, the detailed explanation of determining the disease classification will be described in combination with EATLM model.

In one embodiment, the method 200 as shown in FIG. 2 may be used to implement a classification prediction, e.g., a disease classification prediction. Accordingly, the EATLM model according to embodiments of the present disclosure may be used to perform the method 200 to implement a disease classification prediction related to the antibody sequence based on at least one of: evolution information between the antibody sequence and the germline sequence, or a mutation position on the antibody sequence. Specifically, in some embodiments, the EATLM model according to embodiments of the present disclosure may implement a disease classification prediction related to the antibody sequence based on evolution information between the antibody sequence and the germline sequence. In some embodiments, the EATLM model may implement a disease classification prediction related to the antibody sequence based on a mutation position on the antibody sequence. In some embodiments, the EATLM model may implement a disease classification prediction related to the antibody sequence based on evolution information between the antibody sequence and the germline sequence as well as on a mutation position on the antibody sequence. In some embodiments, the mutation position is a position which an amino acid of the multiple amino acids mutates.

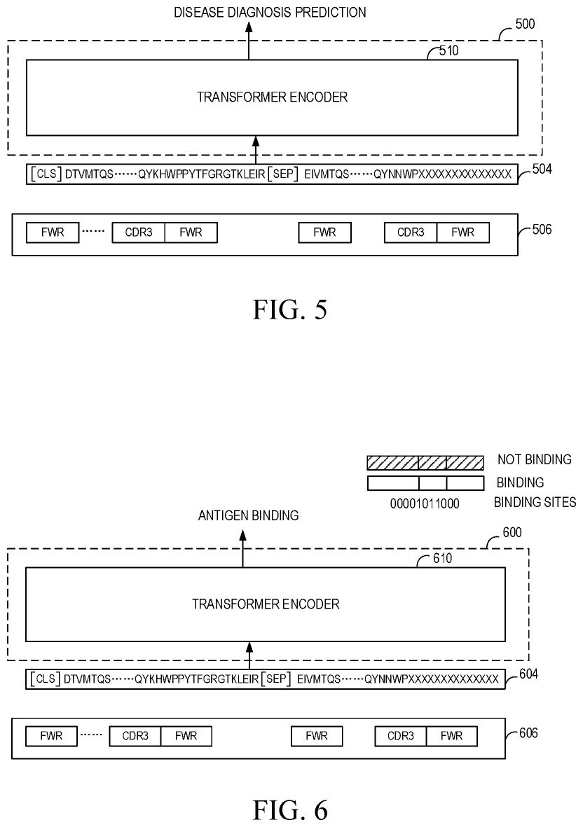

FIG. 5 illustrates an exemplified EATLM model for implementing a disease classification prediction. As shown in FIG. 5, the EATLM model 500 includes a transformer encoder for encoding input of the EATLM model 500. As stated above, the transformer encoder may be implemented according to the existing technology or any technology to be developed in the future. The EATLM model 500 shown in FIG. 5 and the model 300 shown in FIG. 3 are pre-trained in the same way (which will be described in detail below), and fine-tuned in different ways to accommodate respective applications. For example, the model 300 shown in FIG. 3 is fine-tuned in a first way to determine B-cell classification, and the model 500 is fine-tuned in a second way which is different from the first way to implement a disease classification prediction. The detailed process of fine-tuning the models 300 and 500 will described in detail below.

The EATLM model 500 as shown in FIG. 5 may be used to perform the method 200 to implement a classification prediction related to the antibody sequence based on at least one of: evolution information between the antibody sequence and the germline sequence, or a mutation position on the antibody sequence. In some embodiments, the EATLM model 500 as shown in FIG. 5 may implement a classification prediction based on evolution information between the antibody sequence and the germline sequence. In some embodiments, the EATLM model 500 may implement a classification prediction based on a mutation position on the antibody sequence. In some embodiments, the EATLM model 500 may implement a classification prediction based on evolution information between the antibody sequence and the germline sequence as well as on a mutation position on the antibody sequence. The mutation position is a position which an amino acid of the multiple amino acids mutates.

As shown in FIG. 5, the input of the EATLM model 500 may include an antibody sequence and a germline of the antibody sequence. It should be understood that, the number of antibody sequences is not limited in the embodiments of the present disclosure, and may be depended on the actual application and requirements of the EATLM model 500. An example of input antibody sequences and germlines of the antibody sequences is shown in FIG. 5, as labeled by 504. In some embodiments, the input antibody sequences and germlines of the antibody sequences may be descended from a complementarity-determining region (e.g., CDR3) and/or a framework region (FWR) as shown in in FIG. 5, as labeled by 506. The EATLM model 500 may implement a disease classification prediction related to the antibody sequence based on at least one of: evolution information between the antibody sequence and the germline sequence, or a mutation position on the antibody sequence.

In some embodiments, the antibody sequence is from an individual (with the consent of the individual), and the prediction result related to the antibody sequence is a probability of a disease classification. In some embodiments, the EATLM model 500 is implemented on an electronic device. The electronic device may obtain a group of antibody sequences including the antibody sequence. The electronic device may also obtain a group of germline sequences including the germline sequence. The electronic device determines multiple probabilities for the group of antibody sequences respectively based on the group of antibody sequences and the group of germline sequences. Specifically, the electronic device may input each antibody sequence and corresponding germline sequence to the EATLM model 500, and the EATLM model 500 may determine a probability for each antibody sequence based on at least one of: evolution information between the antibody sequence and the germline sequence, or a mutation position on the antibody sequence. In this way, the electronic device determines multiple probabilities for the group of antibody sequences respectively. The electronic device may determine a trimmed mean of the probabilities over the group of antibody sequences to get a score for the individual. And the score is determined by the electronic device as a probability of a disease classification. In some embodiments, the EATLM model 500 may be fine-tuned for a particular disease classification to determine the probability of the particular disease classification relate to the individual.

In some embodiments, the prediction result related to the antibody sequence may include a probability that an amino acid in the antibody sequence binds with an antigen. Accordingly, the EATLM model may be used to perform the method 200 to determine a probability that an amino acid in the antibody sequence binds with an antigen based on at least one of: evolution information between the antibody sequence and the germline sequence, or a mutation position on the antibody sequence. In the following description, the detailed explanation of determining a probability that an amino acid in the antibody sequence binds with an antigen will be described in combination with the EATLM model.

In one embodiment, the method 200 as shown in FIG. 2 may be used to implement a determination of a probability that an amino acid in the antibody sequence binds with an antigen. Accordingly, the EATLM model according to embodiments of the present disclosure may be used to perform the method 200 to determine a probability that an amino acid in the antibody sequence binds with an antigen based on at least one of: evolution information between the antibody sequence and the germline sequence, or a mutation position on the antibody sequence. Specifically, in some embodiments, the EATLM model according to embodiments of the present disclosure may determine a probability that an amino acid in the antibody sequence binds with an antigen based on evolution information between the antibody sequence and the germline sequence. In some embodiments, the EATLM model may determine a probability that an amino acid in the antibody sequence binds with an antigen based on a mutation position on the antibody sequence. In some embodiments, the EATLM model may determine a probability that an amino acid in the antibody sequence binds with an antigen based on evolution information between the antibody sequence and the germline sequence as well as on a mutation position on the antibody sequence. In some embodiments, the mutation position is a position which an amino acid of the multiple amino acids mutates.

FIG. 6 illustrates an exemplified EATLM model for implementing antigen binding prediction. As shown in FIG. 6, the EATLM model 600 includes a transformer encoder for encoding input of the EATLM model 600. As stated above, the transformer encoder may be implemented according to the existing technology or any technology to be developed in the future. The EATLM model 600 shown in FIG. 6, the model 300 shown in FIG. 3, and the model 500 shown in FIG. 5 are pre-trained in the same way (which will be described in detail below), and fine-tuned in different ways to accommodate respective applications. For example, the model 300 shown in FIG. 3 is fine-tuned in a first way to determine B-cell classification, the model 500 is fine-tuned in a second way which is different from the first way to implement a disease classification prediction, and the model 600 shown in FIG. 6 is fine-tuned in a third way that is different from the first way and the second way to determine a probability that an amino acid in the antibody sequence binds with an antigen.

The EATLM model 600 as shown in FIG. 6 may be used to perform the method 200 to determine a probability that an amino acid in the antibody sequence binds with an antigen based on at least one of: evolution information between the antibody sequence and the germline sequence, or a mutation position on the antibody sequence. In some embodiments, the EATLM model 600 as shown in FIG. 6 may determine a probability that an amino acid in the antibody sequence binds with an antigen based on evolution information between the antibody sequence and the germline sequence. In some embodiments, the EATLM model 500 may determine a probability that an amino acid in the antibody sequence binds with an antigen based on a mutation position on the antibody sequence. In some embodiments, the EATLM model 600 may determine a probability that an amino acid in the antibody sequence binds with an antigen based on evolution information between the antibody sequence and the germline sequence as well as on a mutation position on the antibody sequence. The mutation position is a position which an amino acid of the multiple amino acids mutates.

As shown in FIG. 6, the input of the EATLM model 600 may include an antibody sequence and a germline of the antibody sequence. It should be understood that, the number of antibody sequences are not limited in the embodiments of the present disclosure, and may be depended on the actual application and requirements of the EATLM model 600. An example of input antibody sequences and germlines of the antibody sequences is shown in FIG. 6, as labeled by 604. In some embodiments, the input antibody sequences and germlines of the antibody sequences may be descended from a complementarity-determining region (e.g., CDR3) and/or a framework region (FWR) as shown in in FIG. 6, as labeled by 606. The EATLM model 600 may predict a probability that an amino acid in the antibody sequence binds with an antigen based on at least one of: evolution information between the antibody sequence and the germline sequence, or a mutation position on the antibody sequence.

Specifically, the EATLM model 600 may compare the ancestor germline sequence and the antibody sequence, and determine the probability that an amino acid in the antibody sequence binds with the antigen based on the comparison and the evolution information. In some embodiment, each probability corresponds to each amino acid in the antibody sequence. The electronic device may compare a determined probability with a predetermined value. If the determined probability is greater than the predetermined value, the electronic may determine that the corresponding amino acid in the antibody sequence binds with the antigen. FIG. 6 shown examples of two antibody sequences: an antibody sequence with at least one amino acid binding with the antigen (the blocks without filling patterns), and an antibody sequence without any amino acid binding with the antigen (the blocks with filling patterns). In addition, for the antibody sequence with at least one amino acid binding the antigen, the model 600 may also output binding sites with a binary sequence, for example, “00001011000” as shown in FIG. 6. In some embodiments, digital “0” indicate a position where an amino acid does not bind with antigen, and digital “1” indicates a position wherein an amino acid binds with antigen. It should be understood that, the indication of a binding site is only exemplified, and any other ways to indicate a binding site may also be employed. In some embodiments, the EATLM model 600 may be fine-tuned for a particular disease to determine the probability of an amino acid binding with the antigen.

The prediction result related to the antibody sequence may include prediction result other than the embodiments as described above. Accordingly, the EATLM model according to embodiments of the present disclosure may be fine-tuned to accommodate respective prediction results. The EATLM model according to embodiments of the present disclosure explores and utilizes the evolution information between the antibody sequence and its ancestor germline sequence. Therefore, the prediction result can be significantly improved. In addition, the EATLM model may be fine-tuned in different ways to accommodate respective prediction results. Therefore, the scientific discovery of clinically useful antibodies against viruses can be facilitated.

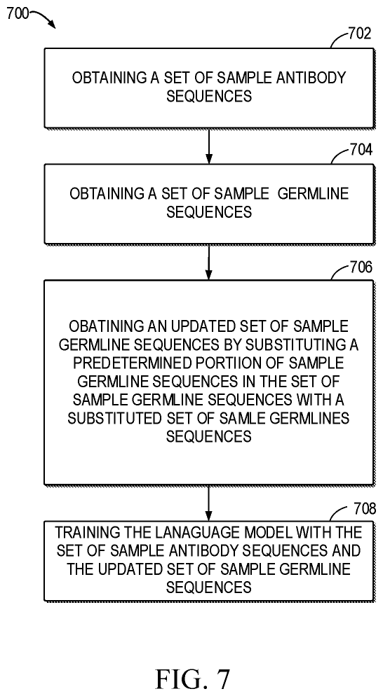

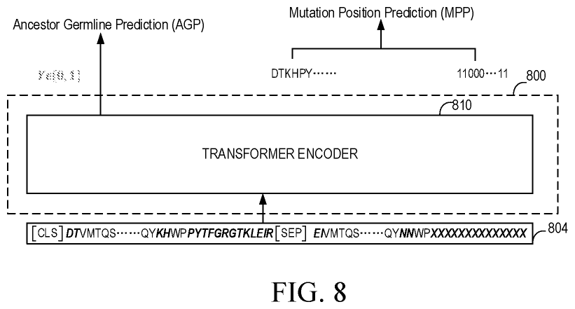

In some embodiments, the EATLM model according to the present disclosure is pre-trained in the first stage. The models 300, 500, and 600 for determining respective prediction result may be pre-trained in the same way in the first stage, and fine-tuned in respective ways in the second stage for determining respective prediction results. In the below, the process of pre-training the EATLM model (for example, models 300, 500, or 600) will be described with reference to FIGS. 7-8.

FIG. 7 illustrates a flowchart of a method 700 for training the EATLM model according to the present disclosure. The method 700 may be implemented by the electronic device which implements the method 200 as shown in FIG. 2. Alternatively, the method 700 may be implemented by another electronic device, and the pre-trained EATLM model may be transmitted to the electronic device for implementing the method 200 as shown in FIG. 2. In some embodiment, the EATLM is implemented by a language model, and the language model being trained for implementing the EATLM model may include any existing model, for example, Bidirectional Encoder Representations from Transformers (BERT). The model may include any further developed models that are suitable for implementing the method according to embodiments of the present disclosure. In addition, it should be appreciated that some of the steps in method 700 may be combined, omitted, performed in parallel, or performed in a different sequence, without affecting the functions achieved.

In FIG. 7, at 702, the electronic device which trains the EATLM model (e.g., BERT) may obtain a set of sample antibody sequences. Each of the sample antibody sequence may be represented by Aₛ={aₛ₁, aₛ₂, . . . , aₛₘ}, and m is an integer indicating the length of the sample antibody sequence.

At 704, the electronic device which trains the EATLM model (e.g., BERT) may obtain a set of sample germline sequences. Each of the sample germline sequences be represented by Gₛ={gₛ₁, gₛ₂, . . . , gₛₙ}, and n is an integer indicating the length of the sample antibody sequence. In some embodiments, the set of sample germline sequences are ancestors of the sample antibody sequences respectively. Then a paired sample sequences Sₛ=(Aₛ, Gₛ)={aₛ₁, aₛ₂, . . . , aₛₘ [SEp]gₛ₁, gₛ₂, . . . , gₛₙ}={sₛ₁, sₛ₂, . . . , sₛₘ₊ₙ₊₁} with a token ‘[SEP]’ as a delimiter.

At 706, the electronic device may obtain an updated set of sample germline sequences by substituting a predetermined portion of sample germline sequences in the set of sample germline sequences with a substituted set of sample germlines sequences. In some embodiments, the substituted set of sample germline sequences are not ancestors of the set of sample antibody sequences. In some embodiments, the predetermined portion may be 30% of the portion. It may be noted that, depending on different requirements, other predetermined portions may be used. Then the electronic device may obtain the updated set of sample germline sequences with a predetermined portion of the sample germline sequences being substituted. Accordingly, for each substituted sample germline sequence, when in combination with the sample antibody sequence, an updated paired sample sequences is S′ₛ=(Aₛ, G′ₛ)={aₛ₁, aₛ₂, . . . , aₛₘ [SEp]g′ₛ₁, g′ₛ₂, . . . , g′ₛₙ}={s′ₛ₁, s′ₛ₂, . . . , s′ₛₘ₊ₙ₊₁}.

FIG. 8 illustrates a language model for a pre-training process according to embodiments of the present disclosure. The input of the language model 800 is a sample antibody sequence and a sample germline sequence, labeled by input sample sequences 804. In combination with the step 706, the original sample germline sequence in the input sample sequences 804 has been substituted by the sample germline sequence as shown in FIG. 8. From FIG. 8, the substituted germline sequence has different amino acids as shown in bold format, e.g., the amino acids Glutamate (E), Isoleucine (I), Asparagine (N) are the different amino acids in the substituted germline sequence from those in the original germline sequence. In addition, amino acids in the input sample antibody sequence may be masked. The masked amino acids are shown in bold format, such as amino acids aspartic acid (D) and Valine (V) are masked for training the model.

Referring back to FIG. 7, at 708, the electronic device trains the language model with the set of sample antibody sequences and the updated set of sample germline sequences. In order to train the model, one or more amino acids in the input sample antibody sequence may be masked for the model to predict the masked antibody sequence. The output of the model 800 includes training ancestor germline prediction information, which may include a trained classification indicating if the sample antibody sequence belongs to the paired sample germline sequence. The classification may help the model to distinguish the ancestor germline of the antibody by capturing the shared features. The output of the model 800 may further include training mutated position information which include the mutated position where an amino acid mutates, and the training mutated amino acid.

As shown in FIG. 8, the model 800 is trained to output training ancestor germline prediction information, which may include a trained classification indicating if the sample antibody sequence belongs to the paired sample germline sequence. For example, Y∈{0,1} indicates a classification of sample antibody sequence. Specifically, digital 0 indicates that the sample germline is not the ancestor of the input sample antibody sequence, and digital 1 indicates that the sample germline is the ancestor of the input sample antibody sequence.

The model 800 further output training mutated position information including the mutated position where an amino acid mutates, and a training mutated amino acid. As shown in FIG. 8, the training mutated position information includes the mutated amino acids “DTKHPY . . . ” and the training mutated position indicated by a binary format “11000 . . . 11”, with digital 1 indicating a mutation position.

In some embodiments, the loss for training the model may be represented as the following Equation 1:

l=l_{MLM}+lₐ+lₘ (Equation 1)

la is the loss for ancestor germline prediction and lₘ is the loss for mutation position prediction. l_{MLM} is the loss for the model itself.

l_{MLM} may be determined according to the following equation 2:

$${l}_{MLM} = - \frac{1}{\vert M \vert} {\sum }_{i ∈ M} log p ( {s}_{i} \vert S \backslash M ) \qquad ( Equation 2 )$$

M is the index set of masked tokens, and \M is the sample antibody sequence in the set of antibody sequences that has not been masked. sᵢ is the predicted masked antibody sequence.

lₐ may be determined according to the following equation 3

lₐ=−log p(y|S′) (Equation 3)

where y∈{0, 1} indicate whether the substituted sample germline sequence is the ancestor of the sample antibody sequence.

lₘ may be determined according to the following equation 4

$${l}_{m} = - \frac{1}{n} {\sum }_{j ∈ \{ 1 , . . . , n \}} log p ( {y}_{j} \vert S \backslash {M}^{′} ) - \frac{1}{\vert M \vert} {\sum }_{i ∈ {M}^{′}} log p ( {a}_{i} \vert S \backslash {M}^{′} ) \qquad ( Equation 4 )$$

M′ is the ground-truth mutation position. By optimizing the loss shown in equation 4, the model learns to capture the specificity obtained from the somatic.

The above described the pre-training process of the model. In some embodiments, the present disclosure may further include fine-tuning of the model to accommodate the reactive prediction results. For example, the ground-truth for fine-tuning may be the true classification of the B-cell when the prediction result is the B-cell analysis. The ground-truth for fine-tuning may be the disease classification when the prediction result is disease classification prediction, and the ground-truth for fine-tuning may be the binding of an amino acid when the prediction result is the probability that an amino acid binds with an antigen. The fine-tuning of the model may be implemented in any suitable ways, and the detailed explanations are omitted here for clarity.

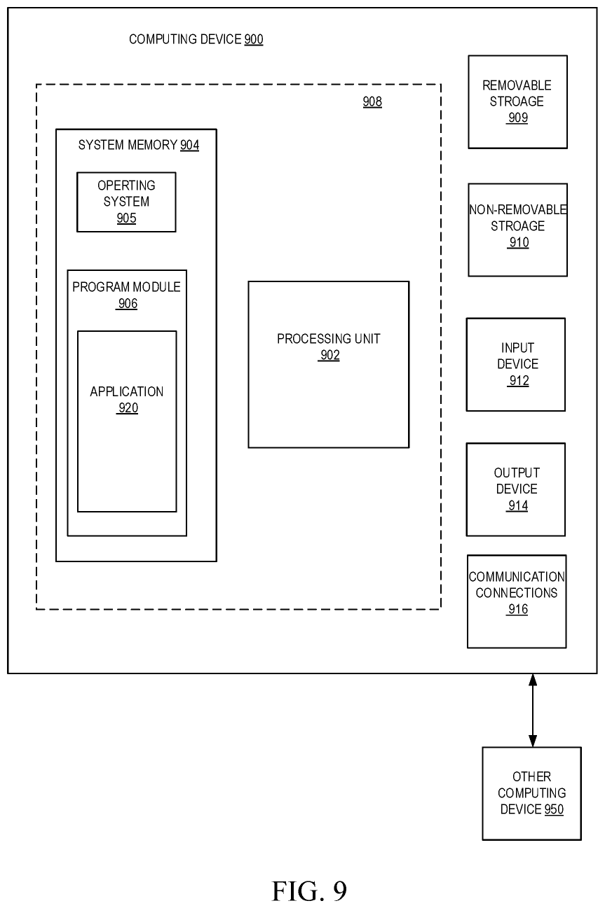

FIG. 9 is a block diagram illustrating physical components (e.g., hardware) of an electronic device 900 with which aspects of the disclosure may be practiced. For example, the electronic device 900 may implement the processes as depicted in FIGS. 1-8. In a basic configuration, the processing device 900 may include at least one processing unit 902 and a system memory 904. Depending on the configuration and type of computing device, the system memory 904 may comprise, but is not limited to, volatile storage (e.g., random access memory), non-volatile storage (e.g., read-only memory), flash memory, or any combination of such memories.

The system memory 904 may include an operating system 905 and one or more program modules 906 suitable for performing the various aspects disclosed herein such. The operating system 905, for example, may be suitable for controlling the operation of the processing device 900. Furthermore, aspects of the disclosure may be practiced in conjunction with other operating systems, or any other application program and is not limited to any particular application or system. This basic configuration is illustrated in FIG. 9 by those components within a dashed line 908. The processing device 900 may have additional features or functionality. For example, the processing device 900 may also include additional data storage devices (removable and/or non-removable) such as, for example, magnetic disks, optical disks, or tape. Such additional storage is illustrated in FIG. 9 by a removable storage device 909 and a non-removable storage device 910.

As stated above, several program modules and data files may be stored in the system memory 904. While executing on the at least one processing unit 902, an application 920 or program modules 906 may perform processes including, but not limited to, one or more aspects, as previously described in more detail with regard to FIGS. 1-8. The program module may include one or more components supported by the systems described herein for performing processes including, but not limited to, one or more aspects, as previously described in more detail with regard to FIGS. 1-8.

Furthermore, aspects of the disclosure may be practiced in an electrical circuit comprising discrete electronic elements, packaged or integrated electronic chips containing logic gates, a circuit utilizing a microprocessor, or on a single chip containing electronic elements or microprocessors. For example, aspects of the disclosure may be practiced via a system-on-a-chip (SOC) where each or many of the components illustrated in FIG. 9 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the capability of determining a prediction result related to the antibody sequence may be operated via application-specific logic integrated with other components of the processing device 900 on the single integrated circuit (chip). Aspects of the disclosure may also be practiced using other technologies capable of performing logical operations such as, for example, AND, OR, and NOT, including but not limited to mechanical, optical, fluidic, and quantum technologies. In addition, aspects of the disclosure may be practiced within a general-purpose computer or in any other circuits or systems.

The processing device 900 may also have one or more input device(s) 912 such as a keyboard, a mouse, a pen, a sound or voice input device, a touch or swipe input device, etc. The output device(s) 914 such as a display, speakers, a printer, etc. may also be included. The aforementioned devices are examples and others may be used. The processing device 900 may include one or more communication connections 916 allowing communications with other computing or processing devices 950. Examples of suitable communication connections 916 include, but are not limited to, radio frequency (RF) transmitter, receiver, and/or transceiver circuitry; universal serial bus (USB), parallel, and/or serial ports.

The term computer readable media as used herein may include computer storage media. Computer storage media may include volatile and nonvolatile, removable and non-removable media implemented in any method or technology for storage of information, such as computer readable instructions, data structures, or program modules. The system memory 904, the removable storage device 909, and the non-removable storage device 910 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the processing device 900. Any such computer storage media may be part of the processing device 900. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

In addition, the aspects and functionalities described herein may operate over distributed systems (e.g., cloud-based computing systems), where application functionality, memory, data storage and retrieval and various processing functions may be operated remotely from each other over a distributed computing network, such as the Internet or an intranet. User interfaces and information of various types may be displayed via on-board computing device displays or via remote display units associated with one or more computing devices. For example, user interfaces and information of various types may be displayed and interacted with. Interaction with the multitude of computing systems with which embodiments of the invention may be practiced include, keystroke entry, touch screen entry, voice or other audio entry, gesture entry where an associated computing device is equipped with detection (e.g., camera) functionality for capturing and interpreting user gestures for controlling the functionality of the computing device, and the like.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

Any of the steps, functions, and operations discussed herein can be performed continuously and automatically. Any of the steps, functions, and operations discussed herein may be combined, omitted, performed in parallel, or performed in a different sequence, without affecting the functions achieved.

The exemplary systems and methods of this disclosure have been described in relation to computing devices. However, to avoid unnecessarily obscuring the present disclosure, the preceding description omits several known structures and devices. This omission is not to be construed as a limitation. Specific details are set forth to provide an understanding of the present disclosure. It should, however, be appreciated that the present disclosure may be practiced in a variety of ways beyond the specific detail set forth herein.

Furthermore, while the exemplary aspects illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, communication device, or collocated on a particular node of a distributed network, such as an analog and/or digital telecommunications network, a packet-switched network, or a circuit-switched network. It will be appreciated from the preceding description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

While the flowcharts have been discussed and illustrated in relation to a particular sequence of events, it should be appreciated that changes, additions, and omissions to this sequence can occur without materially affecting the operation of the disclosed configurations and aspects.

Several variations and modifications of the disclosure can be used. It would be possible to provide for some features of the disclosure without providing others.

In yet another configurations, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another configuration, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another configuration, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure may be implemented as a program embedded on a personal computer such as an applet, JAVA® or CGI script, as a resource residing on a server or computer workstation, as a routine embedded in a dedicated measurement system, system component, or the like. The system may also be implemented by physically incorporating the system and/or method into a software and/or hardware system.

The disclosure is not limited to standards and protocols if described. Other similar standards and protocols not mentioned herein are in existence and are included in the present disclosure. Moreover, the standards and protocols mentioned herein, and other similar standards and protocols not mentioned herein are periodically superseded by faster or more effective equivalents having essentially the same functions. Such replacement standards and protocols having the same functions are considered equivalents included in the present disclosure.

The present disclosure, in various configurations and aspects, includes components, methods, processes, systems and/or apparatus substantially as depicted and described herein, including various combinations, subcombinations, and subsets thereof. Those of skill in the art will understand how to make and use the systems and methods disclosed herein after understanding the present disclosure. The present disclosure, in various configurations and aspects, includes providing devices and processes in the absence of items not depicted and/or described herein or in various configurations or aspects hereof, including in the absence of such items as may have been used in previous devices or processes, e.g., for improving performance, achieving ease, and/or reducing cost of implementation.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

1. A method comprising:
obtaining an antibody sequence comprising a plurality of amino acids;
obtaining a germline sequence of the antibody sequence; and
determining a prediction result related to the antibody sequence based on at least one of: evolution information between the antibody sequence and the germline sequence, or a mutation position on the antibody sequence, wherein an amino acid of the plurality of amino acids mutates on the mutation position.

2. The method of claim 1, wherein the prediction result comprises a category to which a B-cell related to the antibody sequence belongs, and the category is selected from at least one of: immature B-cell, transitional B-cell, mature B-cell, plasmacytes PC, memory IgD−, or memory IgD+.

3. The method of claim 1, wherein the antibody sequence is from an individual, and the prediction result related to the antibody sequence comprises a probability of a classification related to the individual.

4. The method of claim 3, further comprising:
obtaining a group of antibody sequences comprising the antibody sequence;
obtaining a group of germline sequences comprising the germline sequence;
determining a plurality of probabilities for the group of antibody sequences respectively based on the group of antibody sequences and the group of germline sequences; and
determining a trimmed mean of the probabilities over the group of antibody sequences to get a score for the individual.

5. The method of claim 1, wherein the testing result related to the antibody sequence comprises a probability that an amino acid in the antibody sequence binds with an antigen.

6. The method of claim 1, further comprising:
comparing the antibody sequence and the germline sequence; and
determining the probability that the amino acid in the antibody sequence binds with the antigen based on the comparison and the evolution information.

7. The method of claim 1, wherein the method is performed by a language model, and wherein the language model is trained by:
obtaining a set of sample antibody sequences;
obtaining a set of sample germline sequences that are ancestors of the sample antibody sequences respectively;
obtaining an updated set of sample germline sequences by substituting a predetermined portion of sample germline sequences in the set of sample germline sequences with a substituted set of sample germlines sequences, wherein the substituted set of sample germline sequences are not ancestors of the set of sample antibody sequences; and
training the language model with the set of sample antibody sequences and the updated set of sample germline sequences.

8. The method of claim 7, wherein at least one amino acid in the sample antibody sequences and at least one amino acid in the updated set of sample germline sequences are masked to output training mutated position information.

9. The method of claim 8, wherein the language model is further trained by:
outputting training ancestor germline prediction information; and
training the model based on a loss, wherein the loss is determined based on the training mutated position information and the training ancestor germline prediction information.

10. The method of claim 8, wherein the training mutated position information comprises an indication of a mutated position.

11. The method of claim 7, wherein the language model is fine-tuned to accommodate to respective prediction results related to the antibody sequence.

12. An electronic device, comprising:
a memory and a processor;
wherein the memory is used to store one or more computer instructions which, when executed by the processor, cause the processor to:
obtain an antibody sequence comprising a plurality of amino acids;
obtain a germline sequence of the antibody sequence; and
determine a prediction result related to the antibody sequence based on at least one of: evolution information between the antibody sequence and the germline sequence, or a mutation position on the antibody sequence, wherein an amino acid of the plurality of amino acids mutates on the mutation position.

13. The electronic device of claim 12, wherein the prediction result comprises a category to which a B-cell related to the antibody sequence belongs, and the category is selected from at least one of: immature B-cell, transitional B-cell, mature B-cell, plasmacytes PC, memory IgD−, and memory IgD+.

14. The electronic device of claim 12, wherein the antibody sequence is from an individual, and the prediction result related to the antibody sequence comprises a probability of a classification related to the individual.

15. The electronic device of claim 14, wherein the instructions further cause the processor to:
obtain a group of antibody sequences comprising the antibody sequence;
obtain a group of germline sequences comprising the germline sequence;
determine a plurality of probabilities for the group of antibody sequences respectively based on the group of antibody sequences and the group of germline sequences; and
determine a trimmed mean of the probabilities over the group of antibody sequences to get a score for the individual.

16. The electronic device of claim 12, wherein the testing result related to the antibody sequence comprises a probability that an amino acid in the antibody sequence binds with an antigen.

17. The electronic device of claim 12, wherein the instructions further cause the processor to:
compare the antibody sequence and the germline sequence; and
determine the probability that the amino acid in the antibody sequence binds with the antigen based on the comparison and the evolution information.

18. The electronic device of claim 12, wherein the instructions are implemented by a language model, and wherein the instructions further cause the processor to train the language model by:
obtaining a set of sample antibody sequences;
obtaining a set of sample germline sequences that are ancestors of the sample antibody sequences respectively;
obtaining an updated set of sample germline sequences by substituting a predetermined portion of sample germline sequences in the set of sample germline sequences with a substituted set of sample germlines sequences, wherein the substituted set of sample germline sequences are not ancestors of the set of sample antibody sequences; and
training the language model with the set of sample antibody sequences and the updated set of sample germline sequences.

19. A non-transitory computer-readable medium comprising instructions stored thereon which, when executed by an apparatus, cause the apparatus to perform acts comprising:
obtaining an antibody sequence comprising a plurality of amino acids;
obtaining a germline sequence of the antibody sequence; and
determining a prediction result related to the antibody sequence based on at least one of: evolution information between the antibody sequence and the germline sequence, or a mutation position on the antibody sequence, wherein an amino acid of the plurality of amino acids mutates on the mutation position.

20. The non-transitory computer-readable medium comprising instructions stored thereon which, when executed by an apparatus, cause the apparatus to train the language model by:
obtaining a set of sample antibody sequences;
obtaining a set of sample germline sequences that are ancestors of the sample antibody sequences respectively;
obtaining an updated set of sample germline sequences by substituting a predetermined portion of sample germline sequences in the set of sample germline sequences with a substituted set of sample germlines sequences, wherein the substituted set of sample germline sequences are not ancestors of the set of sample antibody sequences; and
training the language model with the set of sample antibody sequences and the updated set of sample germline sequences.
